(19) United States
(12) Patent Application Publication
(10) Pub. No.: US 2012/0252038 A1
(43) Pub. Date: Oct. 4, 2012

(54) STEROID RECEPTOR ASSAYS

(76) Inventor: David A. Chianese, Ardmore, PA (US)

(21) Appl. No.: 13/432,248

(22) Filed: Mar. 28, 2012

Related U.S. Application Data
(60) Provisional application No. 61/470,618, filed on Apr. 1, 2011.

(51) Int. Cl. G01N 33/574

(52) U.S. Cl. 435/7.23

(57) ABSTRACT

Methods for the detection, enumeration and analysis of circulating tumor cells expressing steroid receptors, particularly estrogen receptors are disclosed. These methods are useful for cancer screening and staging, development of treatment regimens, and for monitoring for treatment responses, cancer recurrence or the like. Test kits that facilitate the detection, enumeration and analysis of such circulating tumor cells are also provided.

### FIELD OF THE INVENTION

The present invention relates to the fields of oncology and diagnostic testing, and more particularly to methods for cancer screening and for predicting and monitoring chemotherapy treatment responses, cancer recurrence or the like.

### BACKGROUND OF THE INVENTION

It is estimated that at there are at least several hundred thousand new cases of breast cancer every year. Such cases are currently confirmed by surgical means such as harvesting the tumor or the mastectomy tissue sectioning such tissue into paraffin blocks and using immunohistochemical methods to assess confirm the presence of steroid receptor proteins in such tissue. Due to the surgical procedure, such methods are not used for monitoring the progression of the disease state throughout the course of treatment. Throughout the years, these methods have been improved so that different characteristics of the tissue can be assessed, such as progesterone receptor, and HER/2-neu status, as well as confirming the presence of epitopes of the estrogen receptor. But in all cases this assessment requires a surgical procedure. Further, in the cases of patients whose breast tissue was removed early in their treatment, these methods are simply not available.

Circulating tumor cells (“CTCs”) are present in the blood of patients with metastatic breast cancer and can be harvested throughout a patient's course of treatment. These cells may be analyzed for their HER/2-neu status. However to date, there are no methods to determine the presence of epitopes of the N and C terminal regions of the estrogen receptor of such CTCs. These methods are found in the following invention.

### BRIEF DESCRIPTION OF THE DRAWINGS

FIG. 1 Images of patient samples showing CTCs positive for estrogen receptor α

### DETAILED DESCRIPTION OF THE INVENTION

The invention includes a method for characterizing circulating tumor cells from a patient with metastatic cancer, comprising the steps of:
a) obtaining a biological specimen(blood and bone marrow) from the patient;
b) contacting a the biological specimen is mixed with a ligand that reacts specifically with circulating tumor cells, to the substantial exclusion of other sample components, and permits the separation of such bound circulating tumor cells from other sample components of the biological specimen;
c) contacting the sample of step (b) with at least one reagent that specifically binds the samples of step (b);
d) contacting the sample of step (c) with an agent having binding affinity for steroid receptors in cells; and
e) analyzing the sample to determine the presence of circulating tumor cells expressing steroid receptors

As used herein the term biological samples include but are not limited to whole blood, urine, serum, plasma, sputum, cerebrospinal fluid, amniotic fluid, lavage fluids and bone marrow. The preferred biological samples are whole blood, urine, plasma, sputum, and bone marrow, more preferably, whole blood, urine, plasma, sputum, most preferably whole blood.

The term “ligand” refers to proteins which bind cell surface markers of CTCs, which include but are not limited to adhesion molecules such as E cadherin, N cadherin, as well as monoclonal antibodies to EpCAM, and the like. The preferred ligands include EpCAM antibodies. Further the ligand may comprise “magnetically responsive particles.” Magnetically responsive particles are substances that facilitate magnetic separation. Typically such particles are metallic or organometallic compositions. They may be optionally coated with a polymer, preferably a polymer of biological origin such as BSA. Complexes of the ligand and magnetically responsive particles may be bound to detectable labels which include but are not limited to fluorescent labels. A more detailed description of magnetically responsive particles is found in US. Pat. No. 5,985,153, entitled Magnetic Separation Apparatus and Mehtods Employing an Internal magnetic Capture Gradient and an External Transport Force,” which is hereby incorporated by reference in its entirety.

The term “other sample components” refers to components of the biological samples other than circulating tumor cells. Such components include but are not limited to white blood cells, normal red blood cells, leukocytes, endothelial cells, non-nucleated cells and the like.

The term “reagent” refers to a substance that differentiate between different types of cells. Examples of reagents include but are not limited to dyes such as DAPI, ethidium bromide and acridine orange and probes such as, CD 45, CD41, PAC-1, CD4, CD8 CD56,CD146, CD34, CD38, cytokeratin and the like.

The term “agent” refers to antibodies to particular regions of the steroid receptor. If one is analyzing biological samples for CTCs from an estrogen positive breast cancer patient, antibodies to the N-terminal and C-terminal regions of the estrogen receptor are used. Antibodies to the N-terminal region include but are not limited to ID5, CF11, E115 (rb mono), SP-1 (rb mono), ER119.3 and the like. The preferred N-terminal region antibodies are IDS and E115 (rb mono). Antibodies to the C-terminal region include but are not limited to H222, F10, TE111.5D11. The preferred antibodies to the C-terminal region H222. See M. Pavao et al. Estrogen receptor antibodies: specificity and utility in the detection, localization, and analyses of estrogen receptor α and β; Steroids 66 (2001) 1-16 for other monoclonal antibodies which are useful in this invention.

The term “steroid receptors” has its customary meaning. The preferred steroid receptors are the progesterone, testosterone, and estrogen receptors. The particularly preferred steroid receptors are all subtypes of the estrogen receptor, particularly estrogen receptor α.

Unless otherwise defined herein, scientific and technical terms used in connection with the present invention shall have the meanings that are commonly understood by those of ordinary skill in the art.

The isolation of circulating tumor cells from peripheral blood by immunomagnetic capture with magnetic particles is a multistep process that starts with stabilization and anticoagulation of the blood sample. Next immune magnetic capture and isolation of the target cells with a ferromagnetic particle conjugated to an epithelial specific monoclonal antibody is performed. Fluorescent labeling of the target cells and/or other characterization markers with dyes and fluorescent dye conjugated antibodies specific for the target rare event cell and antigens important to the biology of the cells are used to identify and characterize the cell populations. Finally, cells are visualized by magnetic localization in a cartridge with a window suitable for rare event identification and analysis by fluorescent microscopy, with software that measures the fluorescent signals generated from the microscopic analysis of the target rare event cells. Further, the invention includes a test kit for screening a patient sample for the presence of circulating tumor cells expressing a steroid receptor comprising:
a) a ligand that reacts specifically with circulating tumor cells, to the substantial exclusion of other sample components, and permits the separation of such bound circulating tumor cells from other sample components of the biological specimen;
b) at least one reagent that specifically binds either or both circulating tumor cells and other sample components of a patient
c) an agent having binding affinity for steroid receptors in the circulating tumor cells of the patient.

In order that this invention may be better understood, the following examples are set forth. These examples are for purposes of illustration only and are not to be construed as limiting the scope of the invention in any manner.

### EXAMPLE

Anticoagulation and Preservation of Blood Samples: The commercially available CellSave® tube was used to collect blood samples for this determination. This tube contains sodium EDTA, an anticoagulant, and blood preservative agents which stabilize the target cells in the sample enhancing the assay's ability to detect intact circulating tumor cells. This collection tube facilitates the shipping of samples to distant laboratories for analysis by stabilizing the cells at room temperature for periods of up to 96 hours. Published accounts detail the importance of the use of a preservative to prevent the fragmentation of circulating tumor cells seen in the peripheral blood of patients with solid tissue cancers such as adenocarcinoma of breast. These preservation methods are also compatible with the detection of estrogen receptor protein on these rare event cells.

Ferromagnetic Capture of Target Cells: Ferrofliud bound with an anti-EpCAM antibody clone VU-1D9 was used as part of the commercially available CellSearch® Epithelial Cell kit to localize circulating tumor cells from peripheral blood. This kit enriches rare event cells from as few as five cells in a 7.5 milliliter blood sample containing approximately 30,000,000 to 50,000,000 nucleated blood cells to a working volume of less than 0.5 milliliter with about 2,000 to 3000 leukocytes, and a resulting target cell enrichment of greater than 15,000 times. This reduced volume then allows for staining with fluorescent reagents to identify and characterize the captured rare event cells.

Staining of Captured Cells: The standard CellSearch® kit configuration contains a saponin-based reagent to permeabilize cell and nuclear membranes of intact cells, allowing the labeling of intra-cellular and nuclear proteins. The dye 4′6′diamidinophenylindole is used as an AT base pair, DNA specific dye that fluoresces in the UV range when bound to cell nuclei. This is used by the system to identify nucleated cells present in the isolated sample. Anti-cytokeratin monoclonal antibodies conjugated to fluorescent dyes such as phycoerytherin or fluorescein are used as epithelial specific markers to identify the rare event tumor cells in a population of blood elements. A fluorescent conjugate specific to CD45, a leukocute common antigen, was used to exclude contaminating white blood cells from the analysis.

Estrogen Receptor Specific Reagents: Commercially available monoclonal antibodies to both the N-terminal and C-terminal regions of the estrogen receptor alpha were sourced and conjugated to phycoerytherin and titrated against the cultured MCF-7 breast cancer cell line grown in estrogen depleted culture conditions. Antibody clones H222, SP-1, 1D5 and E115 were shown to work in both the model system of spiked MCF-7 cells in normal donor blood samples. These clones were also demonstrated to be positive in patient samples with advanced breast cancers, as determined on the CellSearch® system.

Analysis: All donor and patient samples were analyzed on the CellTracks® Analyzer II, an automated imaging system that scans cells localized in an analysis chamber as described in U.S. Pat. No. 7,011,794, which is hereby incorporated by reference in its entirety. Images were processed and candidate circulating tumor cell images were presented for operator evaluation. All cells determined to be DAPI and cytokeratin positive, as well as CD45 negative. The cells identified as circulating tumor cells were then scored as being positive or negative for estrogen receptor or other steroid receptor, depending on the reagents used. Nuclear localization of the fluorescent signal was used as a morphologic criterion, matching the analysis used in immunohistochemistry in paraffin sections.

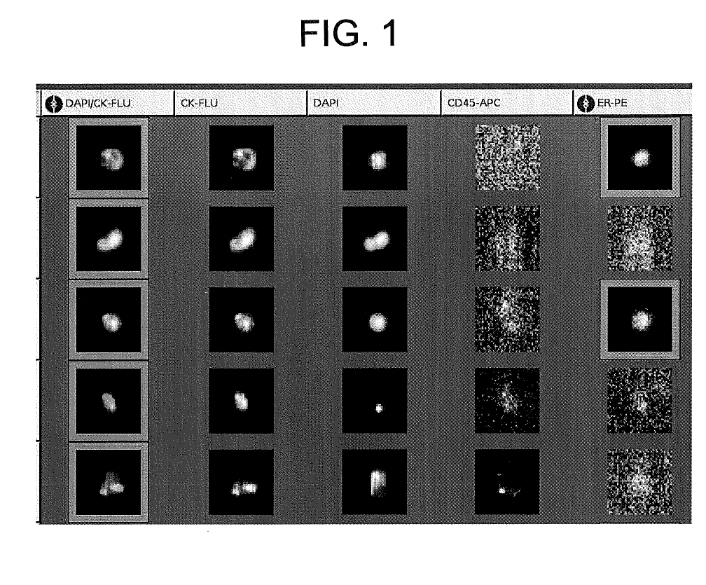

All of the commercial antibodies specific to estrogen receptor alpha evaluated in the model system were shown to be reactive in patient samples. A series of breast cancer patients was evaluated with the E115 rabbit monoclonal (Epitomics Inc.) conjugated to phycoerytherin as well as a fluorescein conjugated monoclonal to HER/2-neu tumor marker as defined by monoclonal antibody Her-81 from the University of Texas. Ten sample runs were performed with donor blood spiked with MCF-7 cells as an assay control. The control samples were all positive for CTC's, with an average of 78% +/−16% of the spiked cells positive for estrogen receptor. The breast cancer patient data is summarized in table 1. FIG. 1 is a gallery of typical CTC images from the CellTracks Analyzer IT® obtained from patient samples

TABLE 1
Summary of a Pilot Series of Breast Cancer Patients

| Breast Cancer Series n = 28 | |
|---|---|
| 9 Patients Positive for CTC | 32% |
| 6 of 9 Samples ER pos | 67% |
| 3 of 9 HER2 pos | 33% |
| 3 of 9 ER/HER2 Dual Positives | 33% |

1. A method for characterizing circulating tumor cells from a patient with metastatic cancer, comprising the steps of:
a) obtaining a biological specimen(blood and bone marrow) from the patient;
b) contacting a the biological specimen is mixed with a ligand that reacts specifically with circulating tumor cells, to the substantial exclusion of other sample components, and permits the separation of such bound circulating tumor cells from other sample components of the biological specimen;
c) contacting the sample of step (b) with at least one reagent that specifically binds the samples of step (b);
d) contacting the sample of step (c) with an agent having binding affinity for steroid receptors in cells; and
e) analyzing the sample to determine the presence of circulating tumor cells expressing steroid receptors

2. The method of claim 1, further comprising the step of determining the number of circulating tumor cells expressing the steroid receptors.

3. The method of claim 1, wherein the agent that reacts specifically with circulating tumor cells is coupled to either or both N-terminal and C-terminal regions of said steroid receptors in said circulating tumor cells.

4. The method of claim 1 wherein said reagent is selected from the group consisting of DAPI, cytokeratin and CD45 neg.

5. The method of claim 1, wherein the ligand binds specifically to an epithelial cell adhesion epitope on the tumor cell.

6. The method of claim 1 wherein the ligand comprises magnetically responsive particles.

7. The method of claim 1, wherein the sample in step (e) is analyzed by at least one process selected from the group consisting of multiparameter flow cytometry, immunofluorescent microscopy, laser scanning cytometry, bright field base image analysis, spectral imaging analysis, manual cell analysis, CELLSTRACKS®analysis, and automated cell analysis.

8. The method of claim 6 further comprising subjecting the product of step (b) to a magnetic field.

9. The method of claim 6, wherein the magnetically responsive particles are colloidal.

10. The method of claim 1, wherein the agent is an antibody selected from the group consisting of ID5, CF11, E115 (rb mono), ER119.3, and SP-1 (rb mono).

11. The method of claim 1 wherein the agent is an antibody selected from the group consisting of H222, F10, and TE111.5D11

12. A test kit for screening a patient sample for the presence of circulating tumor cells expressing a steroid receptor comprising:
a) a ligand that reacts specifically with circulating tumor cells, to the substantial exclusion of other sample components, and permits the separation of such bound circulating tumor cells from other sample components of the biological specimen;
b) at least one reagent that specifically binds either or both circulating tumor cells and other sample components of a patient
c) an agent having binding affinity for steroid receptors in the circulating tumor cells of the patient.
